(19) United States
(12) Patent Application Publication
(10) Pub. No.: US 2019/0079152 A1
(43) Pub. Date: Mar. 14, 2019

(54) MR-VISIBLE MARKER FOR AN MRI APPARATUS AND AN MR GUIDED RADIATION THERAPY SYSTEM

(71) Applicant: KONINKLIJKE PHILIPS N.V., EINDHOVEN (NL)

(72) Inventors: WYCLIFFE ADELL RADUMA, ESPOO (FI); TEEMU NIEMI, VANTAA (FI); TIINA NASI, HELSINKI (FI); PANU TAPANI VESANEN, HELSINKI (FI); JOEL TULPPO, VANTAA (FI)

(21) Appl. No.: 16/084,588

(22) PCT Filed: Mar. 17, 2017
(86) PCT No.: PCT/EP2017/056418

§ 371 Date: Sep. 13, 2018

(30) Foreign Application Priority Data
Mar. 17, 2016 (EP) 16160949.0

(51) Int. Cl. G01R 33/3415; G01R 33/48; G01R 33/58; A61B 5/055

(52) U.S. Cl. CPC G01R 33/3415; G01R 33/4808; A61B 5/0033; A61B 5/055; G01R 33/58

(57) ABSTRACT

The invention relates to a device (34) for an MRI apparatus (14), especially an MRI apparatus (14) of an MR guided radiation therapy system (10), the device (34) comprising at least one MR imaging marker unit (72), wherein the marker unit (72) comprises an at least slightly MR-visible film (70) covering at least one conducting part of an electrical circuit loop (68) of the device (34). The invention further relates to the corresponding MRI apparatus (14) and MR guided radiation therapy system (10).

### FIELD OF THE INVENTION

The invention relates to a device for an MRI (MRI: magnetic resonance imaging) apparatus, especially an MRI apparatus of an MR (MR: magnetic resonance) guided radiation therapy system, the device comprising an MR imaging marker unit including marker agents. The invention further relates to the corresponding MRI apparatus and MR guided radiation therapy system.

### BACKGROUND OF THE INVENTION

The receive coils of the MR guided radiation therapy system are placed as close as possible to the treated and imaged anatomy to maximize image quality and enable the MR guided radiation therapy system to provide efficient MR guidance for the radiation beam. As a consequence, receive coils are located in the radiation beam path and this results that the coils attenuate and may cause non-idealities in the radiation therapy which may need to be taken into account in the delivery of the treatment.

Because the coil attenuates the radiation beam, its location needs to be known accurately so that the radiation dose can be adjusted to compensate for the coil's attenuation. Even if the location of the coil may be known accurately, and even though the coil's attenuation may be insignificant or ignorable, the most accurate treatment requires that all insignificant attenuation obstacles are taken into account, in order to be able to assess their cumulative effect. In MR imaging these markers usually comprise marker units in the form of liquid capsules that are visible in MRI scans. These markers are primarily used to accurately display their spatial location in an MR image. These capsule markers show high radiation attenuation and cannot be switch off (passive markers). Consequently, they can create unwanted image artefacts.

Document US 2012/0224341 A1 shows an MR guided radiation therapy system comprising a radiation emitter and a MRI apparatus, which MRI apparatus includes a device an RF coil and MR imaging markers, which markers can be active or passive marker. An active marker is, e.g., visible only when activated by energizing a marker coil surrounding the marker unit. The use of these active markers reduces the unwanted image artefacts.

US 2015/0031981A1 describes a high frequency antenna. The high frequency antenna includes a layer which at least partially includes an imaging material. This means that it is possible to locate precisely where the high frequency antenna is during a magnetic resonance measurement. The layer containing the imaging material covers at least 50% of a detection area of the high frequency antenna unit.

US 2015/0160310 describes a coil system for an interventional magnetic resonance examination system. The coil system comprises an opening that has the same shape as a penetration template. The coil has a marker element arranged at the opening. The magnetic resonance marker element is fashioned as a channel which is arranged around the opening, along the circumference of the opening. The channel is designed to receive a magnetic resonance visible fluid.

### SUMMARY OF THE INVENTION

It is an object of the invention to provide a device for an MRI apparatus, an MRI apparatus and an MR guided radiation therapy system to reduce unwanted image artefacts even further.

This object is achieved by the features of the independent claims. The dependent claims detail advantageous embodiments of the invention.

According to various embodiments of the invention, the device for an MRI apparatus comprises at least one MR imaging marker unit, which marker unit comprises an (at least slightly) MR-visible film covering at least one conducting part of an electrical circuit loop of the device. In other words, at least those parts of the film being in close proximity to the conducting part of the loop become visible because the film is more or less directly attached to the electrical circuit loop. This relies on the principal that signal from the film is increasing exponentially as the distance to the conducting part decreases. The MRI marker unit is an active marker unit, which is visible only when activated by energizing the electrical circuit loop. In one of the simplest cases, the device is an active marker. These kind of markers can be attached to any kind of subjects and serve as fiducial markers. Preferably, only small parts of the film being in close proximity to the conducting part of the loop become visible when activated so that the at least slightly MR-visible film is a “slightly MR-visible film”.

The device further comprises at least one MR imaging antenna having a predefined distance to the marker unit, such that the MR-visible film is not visible when receiving with the at least one MR imaging antenna alone. The MR imaging antenna is positioned at a certain predefined distance from the MR-visible film, which distance is larger than the distance between the MR visible film and the electrical circuit loop, such that the MR imaging antenna will not pick up the signal from the MR-visible film, at least not to the extent that the MR visible film will cause clinically relevant artifacts in MR images created from the signals received by the MR imaging antenna. In this way imaging artifacts, in particular fold over artifacts, can be reduced. Because the distance between the marker unit and the MR imaging antenna is predefined, a it is possible to derive the position of the MR imaging unit when the position of the MR-visible film is known.

According to embodiments of the invention, the device is configured such that the MR imaging marker unit can be switched off independently of the at least one MR imaging antenna

According to a preferred embodiment of the invention, the at least one MR imaging marker unit is essentially transparent for the radiation used by the radiation therapy system for radiation therapy, especially x-ray transparent.

According to another preferred embodiment of the invention, the device further comprises at least one MR imaging antenna having a predefined distance to the marker unit. The MR imaging antenna comprises a coil (with a plurality of conductor loops) or at least one conductor loop.

According to yet another preferred embodiment of the invention the marker unit is located inside the loop of the MR imaging antenna or at least one of the MR imaging antennas.

According to yet another preferred embodiment of the invention the device comprises a plurality of MR imaging marker units. Preferably each of the MR-visible films is covering one individual conducting part of the one electrical circuit loop.

According to another preferred embodiment of the invention, the device further comprises a basic body supporting the electrical circuit loop and/or the slightly MR-visible film. Preferably the basic body further supports the at least one MR imaging antenna.

According to yet another preferred embodiment of the invention the basic body is a printed circuit board.

According to a preferred embodiment of the invention the electrical circuit loop and the MR imaging antenna are arranged for connecting to one common preamplifier.

According to another preferred embodiment of the invention the electrical circuit loop on the one hand and the loop of the MR imaging antenna or at least one of the loops of the MR imaging antennas on the other hand have an electrically conductive interconnection with each other.

According to yet another preferred embodiment of the invention the electrical circuit loop is arranged for use as an MR imaging antenna. Preferably the device comprises two electrical circuit loops, each being arranged for use as an MR imaging antenna. In this case the one loop can be used as an MR imaging antenna and the other loop can be used together with the slightly MR-visible film to form the marker unit. Said marker unit can be used as a fiducial marker for the MR imaging antenna.

According to various embodiments of the invention, the MRI apparatus comprises an aforementioned device. The MRI apparatus especially is an MR apparatus for an MR guided radiation therapy system like an MR-guided linear accelerator system.

According to various embodiments of the invention, the MR guided radiation therapy system comprises a radiation emitter and an aforementioned MRI apparatus for guiding the radiation beam of said radiation emitter.

### BRIEF DESCRIPTION OF THE DRAWINGS

These and other aspects of the invention will be apparent from and elucidated with reference to the embodiments described hereinafter.

In the drawings:

FIG. 1 shows an MR-guided radiation therapy system according to a preferred embodiment of the invention;

FIG. 2 shows an embodiment of a device for an MRI apparatus of the MR-guided radiation therapy system;

FIG. 3 shows another embodiment of the device for the MRI apparatus;

FIG. 4 shows another embodiment of the device for the MRI apparatus; and

FIG. 5 shows yet another embodiment of the device for the MRI apparatus.

### DETAILED DESCRIPTION OF EMBODIMENTS

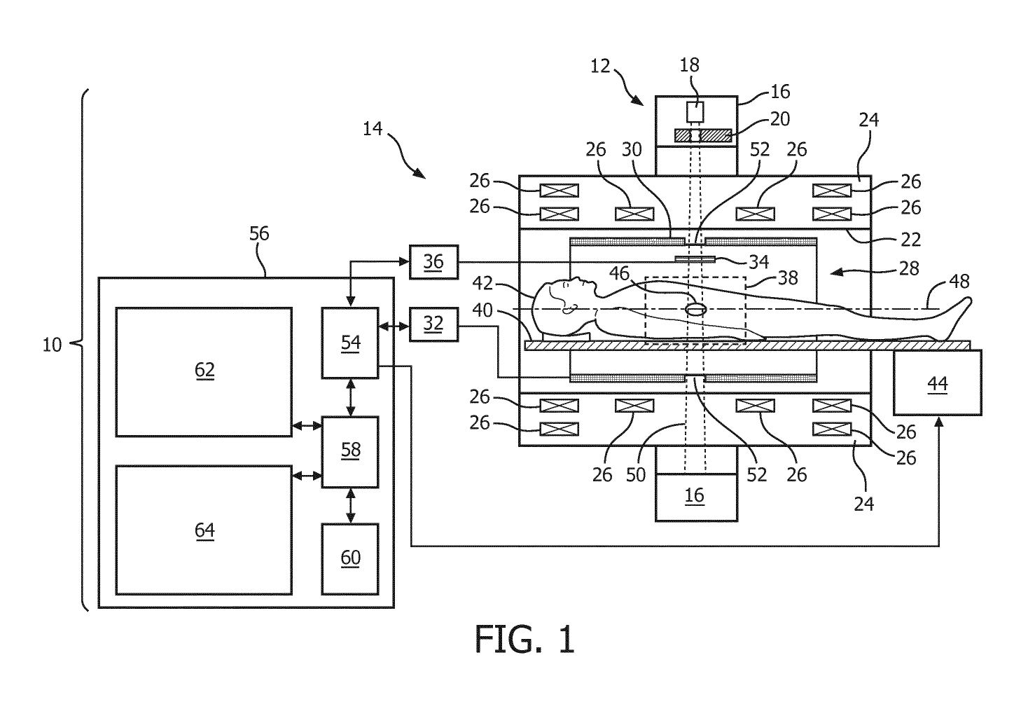

FIG. 1 shows an embodiment of an MR-guided radiation therapy system 10 according to the invention. The MR-guided radiation therapy system 10 comprises a LINAC 12 and a magnetic resonance imaging device (MRI device) 14. The LINAC 12 comprises a gantry 16 and a X-ray source 18. The gantry 16 is for rotating the X-ray source 18 about an axis of gantry rotation 20. Adjacent to the X-ray source 18 is an adjustable collimator 20. The adjustable collimator 20 may for instance have adjustable plates for adjusting the beam profile of the X-ray source 18. The adjustable collimator 20 may, for example, be a multi-leaf collimator. The magnetic resonance imaging device 14 comprises a magnet 22.

It is also possible to use permanent or resistive magnets. The use of different types of magnets is also possible for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject: the arrangement of the two sections area similar to that of a Helmholtz coil. Open magnets are popular, because the subject is less confined. Inside the cryostat of the cylindrical magnet there is a collection of superconducting coils. The magnet 22 shown in this embodiment is a standard cylindrical superconducting magnet. The magnet 22 has a cryostat 24 with superconducting coils 26 within it. The magnet 22 has a bore 28. Within the bore 28 of the cylindrical magnet 22 there is an imaging zone where the magnetic field is strong and uniform enough to perform magnetic resonance imaging.

Within the bore 28 of the magnet 22 is a magnetic field gradient coil 30 for acquisition of magnetic resonance data to spatially encode magnetic spins within an imaging zone of the magnet. The magnetic field gradient coil 30 is connected to a magnetic field gradient coil power supply 32. The magnetic field gradient coil 30 is intended to be representative, to allow radiation to pass through without being attenuated it will normally be a split-coil design. Typically magnetic field gradient coils contain three separate sets of coils for spatially encoding in three orthogonal spatial directions. The magnetic field gradient power supply 32 supplies current to the magnetic field gradient coils 30. The current supplied to the magnetic field coils 30 is controlled as a function of time and may be ramped or pulsed.

There is a device 34 connected to a transceiver 36, which device is shown in detail in the following figs. The device is adjacent to an imaging zone 38 of the magnet 22. The imaging zone 38 has a region of high magnetic field and homogeneity which is sufficient for performing magnetic resonance imaging. The device 34 may be for manipulating the orientations of magnetic spins within the imaging zone and for receiving radio transmissions from spins also within the imaging zone. The device 34 may also be referred to as an antenna or channel. The device 34 is intended to also represent a dedicated transmit antenna and a dedicated receive antenna. Likewise the transceiver may also represent a separate transmitter and receivers.

Also within the bore 28 of the magnet 22 is a subject support 40 for supporting a subject 42. The subject support 40 may be positioned by a mechanical positioning system 44. Within the subject 42 there is a target zone 46. An axis of gantry rotation 48 is coaxial in this particular embodiment with the cylindrical axis of the magnet 22. The subject support 40 has been positioned such that the target zone 46 lies on the axis 48 of gantry rotation. The X-ray source 18 is shown as generating a radiation beam 50 which passes through the collimator 20 and through the target zone 46. As the radiation source 18 is rotated about the axis 48 the target zone 46 will always be targeted by the radiation beam 50. The radiation beam 50 passes through the cryostat 24 of the magnet. The magnetic field gradient coil 30 has a gap 52 which separate the magnetic field gradient coil 30 into two sections. The gap 52 reduced attenuation of the radiation beam 50 by the magnetic field gradient coil 30. In an alternative embodiment a split or open magnet design is used to reduce the attenuation of the X-ray beam by the magnet 22. The device 34 can be seen as being attached to the inside of the bore of the magnet 22 (not shown).

The transceiver 36, the magnetic field gradient coil power supply 32 and the mechanical positioning system 44 are all shown as being connected to a hardware interface 54 of a computer system 56. The computer system 56 is shown as further comprising a processor 58 for executing machine executable instructions and for controlling the operation and function of the MR-guided radiation therapy system 10. The hardware interface 54 enables the processor 58 to interact with and control the MR-guided radiation therapy system 10. The processor 58 is shown as further being connected to a user interface 60, computer storage 62, and computer memory 64.

The computer storage 62 contains a treatment plan and an X-ray transmission model of the device 34. The X-ray transmission model may comprise the location of sensitive components of the device 34 and also the X-ray transmission properties of the device 34. The computer storage 62 further contains a pulse sequence. A pulse sequence as used herein is a set of commands used to control various components of the magnetic resonance imaging device 14 to acquire magnetic resonance data. The computer storage 62 contains magnetic resonance data that was acquired using the magnetic resonance imaging device 14.

The computer storage 62 is further shown as containing a magnetic resonance image that was reconstructed from the magnetic resonance data. The computer storage 62 is further shown as containing an image registration of the magnetic resonance image. The image registration registers the location of the image relative to the magnetic resonance imaging device 14 and the LINAC 12. The computer storage 62 is further shown as containing the location of the target zone 46. This was identified in the magnetic resonance image. The computer storage 62 is further shown as containing control signals. The control signals are control signals which are used to control the LINAC 12 to irradiate the target zone 46.

The computer memory 64 is shown as containing a control module. The control module contains computer-executable code which enables the processor 58 to control the operation and function of the medical apparatus 10. For instance the control module may use the pulse sequence to acquire the magnetic resonance data. The control module may also use the control signals to control the LINAC 12. The computer memory 64 is further shown as containing a treatment plan modification module. The treatment plan modification module modifies the treatment plan using the information contained in the X-ray transmission model. The computer memory 64 is shown as further containing an image reconstruction module. The image reconstruction module contains code which enables the processor 58 to reconstruct the magnetic resonance image from the magnetic resonance data.

The computer memory 64 is shown as further containing an image registration module. The image registration module contains code which enables the processor 58 to generate the image registration in the location of the target zone 46 using the magnetic resonance image. The computer memory 64 is shown as further containing a target zone location module. The target zone location module contains code which enables the processor 58 to generate the location of the target zone 46 using the image registration. The computer memory 64 is further shown as containing a control signal generation module. The control signal generation module contains code which enables the processor 58 to generate the control signals from the treatment plan and the location of the target zone. The treatment plan after it has been modified in accordance with the X-ray transmission module is used.

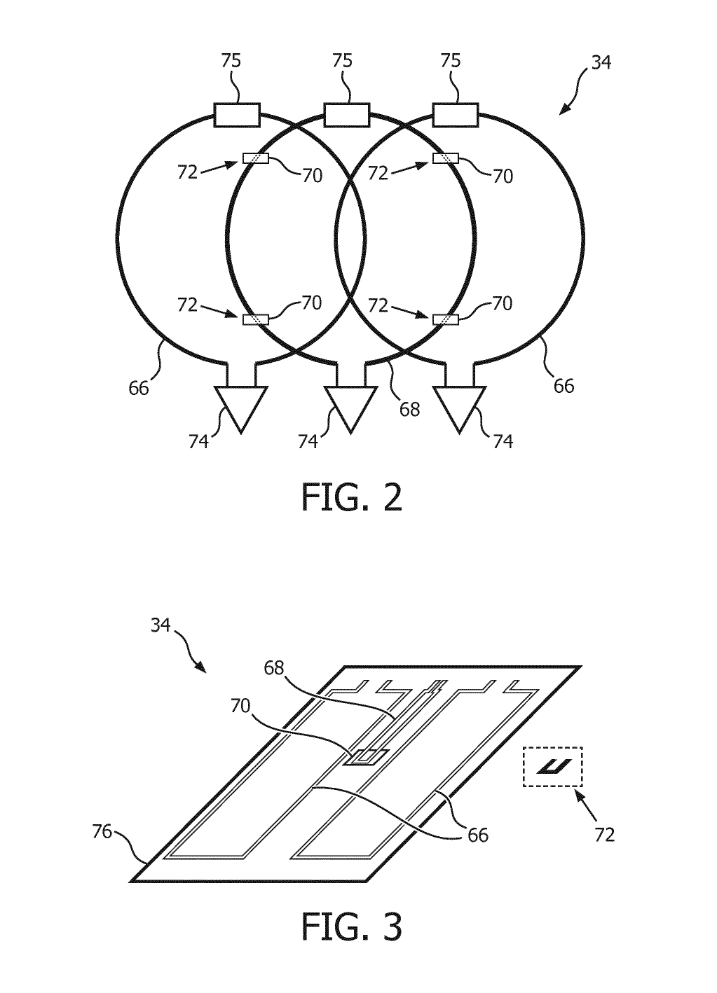

FIG. 2 shows the device 34 for the MRI apparatus 14 of the MR guided radiation therapy system 10 in detail. The device 34 comprises two MR imaging antennas 66, each with a corresponding antenna loop, an electrical circuit loop 68 and 4 MR imaging marker units 72, each unit 72 comprising a slightly MR-visible film 70 covering at least one conducting part of the electrical circuit loop 68. In other words, the device shown in the figs. comprises MR imaging marker units 72, being active markers used as fiducial markers for the MR imaging antennas 66. This kind of device 34 is a MR imaging antenna device with antennas 66 and active fiducial markers. The marker units 72 are visible only when activated by energizing the electrical circuit loop 68. The MR imaging antennas 66 and the maker units 72 are located within the imaging zone 38. Each of the marker units 72 is located inside a corresponding loop of one of the MR imaging antennas 66. The film 70 becomes visible because it is more or less directly attached to the loop 68. This relies on the principal that signal from the film 70 increasing exponentially as the distance to a conductor decreases.

The MR imaging marker unit 72 are essentially transparent for the radiation used by the radiation therapie system 10 for radiation therapie, namely X-ray. This implementation on the x-ray transparent marker units 72 allows the marker units 72 to have a low attenuation of the radiation beam 50. Furthermore, it is possible to implement it in a way that the marker units 72 can be switched off.

The marker switching loop 68 does not need to be isolated from the adjacent loops of the imaging antennas 66 as they are never used for imaging at the same time. The detune lines of the marker switching loop 68 are controlled independently from the detune elements 75 of the normal receive loops of the imaging antennas 66. The film(s) 70 emits very weak MRI signal and is thus visible only to the loop 68 whose copper trace is immediately next to the film(s). Thus the film is not visible when receiving with the two loops of the imaging antennas 66 and does not fold into the volume of interest.

In this embodiment the image registration module is used to detect the location of the marker units 72 in the magnet resonance image to generate accessory locations, which is stored in the computer storage. Each of the MR imaging antennas 66 and the electrical circuit loop 68 is electrically connectable/connected to a corresponding preamplifier 74 and comprises a detune element 75 on the respective coil 66, 68.

FIG. 3 shows a concrete embodiment of device 34. The device 34 further comprises a basic body 76 supporting the MR imaging antennas 66, the electrical circuit loop 68 and the slightly MR-visible film 70. The basic body is realized as a printed circuit board (PCB) with the loops of the MR imaging antennas 66 and the electrical circuit loop 68 being formed as conductor tracks. The loops of the imaging antennas 66 are separate from the marker-switching electrical circuit loop 68.

As already mentioned, the film 70 becomes visible because it is attached to the loop 68. This relies on the principal that signal from the film 70 increasing exponentially as the distance to an antenna's conductor decreases. In FIG. 3, the effect is displayed as a slightly visible adhesive film glued across the surface of a receive coil.'s PCB. The illumined elements are intersections of adhesive film and the antennas' copper conductors as shown on the right side of FIG. 3.

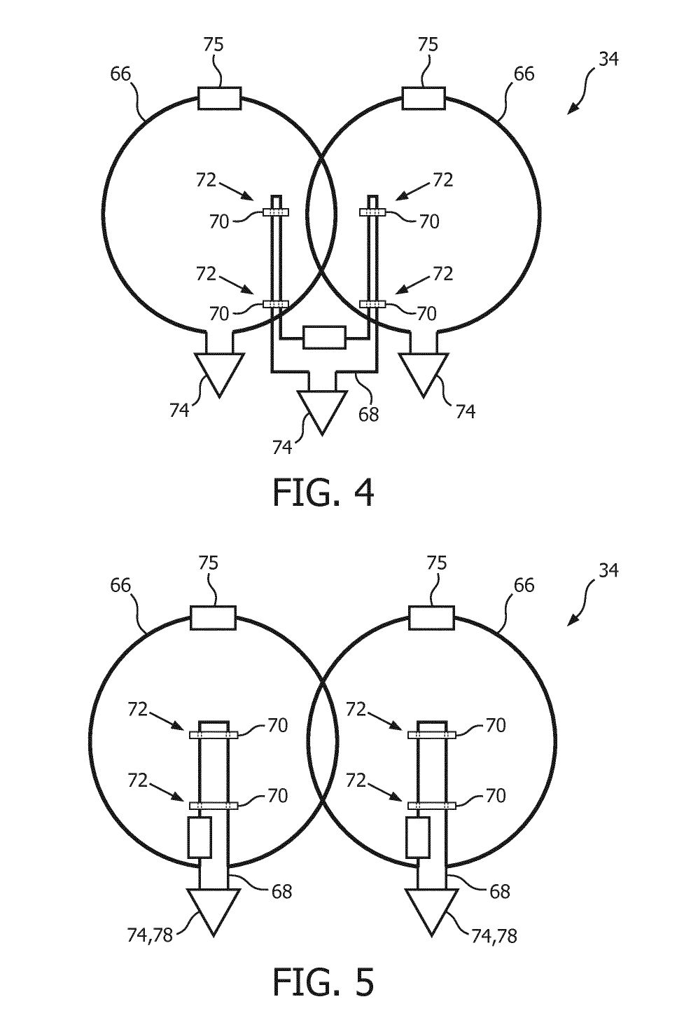

FIG. 4 shows an embodiment slightly different from the embodiment of FIG. 2. In FIG. 4, the surface area of the marker switching loop 68 is minimized to decrease the coupling to the coils of the imaging antennas 66 and thus the need for the detune elements 75 on the coil.

Even though the marker loop 68 is separate from the coils of the antennas 66, it is possible to share a preamplifier 74 with the loop of an imaging antenna, thus avoiding the use of extra channels. In other words: the electrical circuit loop 68 and the MR imaging antenna 66 are connected to one common preamplifier 78. FIG. 5 shows a corresponding embodiment of device 34 with two electrical circuit loops 68. The electrical circuit loop 68 and one of the loops of the MR imaging antennas 66 have an electrically conductive interconnection with each other. Elaborating on this, if there is no need for the makers to be switchable, the tapes can be placed so that they intersect with imaging loops instead of having a dedicated loop (FIG. 5). Nevertheless, switching off of these marker units 70 can be done by imaging with the body coil to hide the marker units 70.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A device for an MRI apparatus, configured for an MR guided radiation therapy system, the device comprising
at least one MR imaging marker unit, wherein the marker unit comprises an MR-visible film covering at least one conducting part of an electrical circuit loop of the device, wherein the MR-visible film is visible when receiving with the electrical circuit loop and wherein the device further comprises
at least one MR imaging antenna having a predefined distance to the marker unit, such that no MR-visible film is visible when receiving with the at least one MR imaging antenna alone.

2. The device of claim 1, wherein the MR imaging marker unit can be switched off independently of the at least one MR imaging antenna.

3. The device of claim 1, wherein the at least one MR imaging marker unit is essentially transparent for the radiation used by the radiation therapy system for radiation therapy.

4. The device of claim 1, wherein the marker unit is located inside a loop of the MR imaging antenna or at least one of the MR imaging antennas.

5. The device of claim 1, comprising a plurality of MR imaging marker units.

6. The device of claim 5, wherein each of the MR-visible films is covering one individual conducting part of the one electrical circuit loop.

7. The device of claim 1, further comprising a basic body supporting the electrical circuit loop and/or the slightly MR-visible film.

8. The device of claim 7, wherein the basic body further supports the at least one MR imaging antenna.

9. The device of claim 7, wherein the basic body is a printed circuit board.

10. The device of claim 3, wherein the electrical circuit loop and the MR imaging antenna are arranged for connecting to one common preamplifier.

11. The device of claim 3, wherein the electrical circuit loop and the loop of the MR imaging antenna or at least one of the loops of the MR imaging antennas have an electrically conductive interconnection with each other.

12. The device of claim 1, wherein the electrical circuit loop is arranged for use as an MR imaging antenna.

13. A magnetic resonance imaging (MRI) apparatus configured for an MR guided radiation therapy system, wherein the MRI apparatus comprises a device according to claim 1.
at least one MR imaging marker unit, wherein the marker unit comprises an MR-visible film covering at least one conducting part of an electrical circuit loop of the device, wherein the MR-visible film is visible when receiving with the electrical circuit loop and wherein the device; and
at least one MR imaging antenna having a predefined distance to the marker unit, such that no MR-visible film is visible when receiving with the at least one MR imaging antenna alone.

14. A magnetic resonance (MR) guided radiation therapy system comprising a radiation emitter and a magnetic resonance imaging (MRI) apparatus according to claim 13 for guiding the radiation beam of said radiation emitter.
